(19) United States
(12) Patent Application Publication
(10) Pub. No.: US 2009/0131874 A1
(43) Pub. Date: May 21, 2009

(54) Device for application of lubricant

(76) Inventors: Thore Maki, Skelleftea (SE); Urban Maki, Linkoping (SE)

Correspondence Address: DILWORTH & BARRESE, LLP, 333 EARLE OVINGTON BLVD., SUITE 702, UNIONDALE, NY, 11553, US

(21) Appl. No.: 11/921,820

(22) PCT Filed: Jun. 2, 2006
(86) PCT No.: PCT/SE2006/000652

§ 371 Date: Dec. 7, 2007

(30) Foreign Application Priority Data
Jun. 9, 2005 (SE) 0501334-7

(51) Int. Cl. A61M 5/00

(52) U.S. Cl. 604/182

(57) ABSTRACT

A device is provided for application of lubricant in an urethra to thereby facilitate insertion of a medical instrument into the urethra. The device (1) has a receptacle (2) having a lubricant-containing space (3) and a gripping part (4) and spout (5) connected to the gripping part (4). The outer end (5a) of the spout (5) is insertable into the urethra of a patient through the urethra orifice and provided with an outlet opening (6) connected to the lubricant-containing space (3) in the receptacle (2) which is grippable and manipulatable by gripping with fingers about the gripping part (4) of the receptacle (2), the gripping part (4) being deformable so that lubricant from the lubricant-containing space (3) can be squeezed out through the outlet opening (6) of the spout (5) by compression of the gripping part (4) with the fingers.

### FIELD OF THE INVENTION AND PRIOR ART

The present invention relates to a device for application of lubricant in a urethra so as to thereby facilitate the insertion of a medical instrument, such as a urethral catheter or an endoscope, into the urethra.

In this description and the subsequent claims, the expression “urethral catheter” is used for designating the type of catheter that is intended to be inserted into a urethra through the urethral orifice in order to draw urine from the urinary bladder of a patient. Other known denominations for such a type of catheter are urine draining catheter, urinary catheter or urethra catheter.

The expression “urethral catheterization” is used in this description for designating the operation that is made in order to draw urine from the urinary bladder of a patient by means of a urethral catheter that is inserted into the urethra of the patient through the urethral orifice. Urethral catheterization is often performed by the patient by his/her own, for instance in home environment.

The expression “the user” will in this description and the subsequent claims refer to the person who performs the application of the lubricant and/or the catheterization. The user may be the patient himself/herself or another person who performs the application of the lubricant and/or the catheterization on the patient, such a doctor, a nurse etc.

Lubricant, for instance in the form of gel, is often used in connection with urethral catheterization in order to facilitate the insertion of the urethral catheter into the urethra. The lubricant is usually applied on the outside of the catheter before the catheter is inserted into the urethra through the urethral orifice. A problem with this procedure is that the outside of the urethral catheter will become smeary by the lubricant, which may be inconvenient to the user and make it more difficult for the user to handle the catheter. Different types of solutions have been suggested in order to avoid this problem. The U.S. Pat. No. 3,854,483 A disclosed for instance a solution where the urethral catheter is mounted in a device with a lubricant-containing space and where the catheter during the insertion into the urethra is pressed through the lubricant-containing space and pushes the lubricant in front of itself into the urethra. The U.S. Pat. No. 5,209,726 A discloses another solution with a specially designed double-walled urethral catheter, which contains lubricant in the space between the walls. In this case, the lubricant is pressed into the urethra via the inner channel of the urethral catheter as the catheter is displaced inwards in the urethra. These solutions are both rather complicated and have not gained any wider use.

### OBJECT OF THE INVENTION

The object of the present invention is to achieve a device which makes a simple and appropriate application of lubricant possible, for instance in connection with urethral catheterization, and which at the same time offers a solution to the above-mentioned problem.

### SUMMARY OF THE INVENTION

According to the present invention, said object is achieved by means of a device having the features defined in claim 1.

The inventive device comprises a receptacle having a lubricant-containing space, which receptacle is provided with a gripping part and a sprout connected to the gripping part, where the outer end of the sprout is dimensioned so as to be insertable into the urethra of a patient through the urethral orifice and is provided with an outlet opening that is connected to the lubricant-containing space in the receptacle. The receptacle is designed to be grippable and manipulatable by a user by gripping with the fingers about the gripping part of the receptacle, the gripping part being deformable so that lubricant from the lubricant-containing space can be squeezed out through the outlet opening of the sprout by compression of the gripping part with the fingers.

With the aid of the inventive device, lubricant may be applied in a urethra of a patient so as to facilitate the insertion into the urethra of a medical instrument, such as for instance a urethral catheter in connection with urethral catheterization or an endoscope in connection with endoscopy.

In connection with the performance of for instance a urethral catheterization, the lubricant is first applied inside the urethra by means of the device in question in that the user inserts the outer end of the sprout of the receptacle into the urethra through the urethral orifice and thereafter with the fingers exerts a pressure against the gripping part so that this is compressed and thereby causes lubricant from the lubricant-containing space to be squeezed into the urethra through the outlet end of the sprout. After the application of the lubricant in the urethra, the receptacle is removed from the urethra, whereupon a urethral catheter in a conventional manner is inserted into the urethra in order to draw urine from the urinary bladder of the patient. The inventive device is very simple to use and eliminates the problem with urethral catheters becoming smeary by lubricant. Owing to the fact that the lubricant is applied inside the urethra, lubricant is also prevented from being scraped off against the urethral orifice in connection with the insertion of the urethral catheter. Furthermore, the device is separate from the urethral catheter during manufacturing as well as during use and can thereby be given a very simple design that makes a cost-effective manufacturing possible.

According to a preferred embodiment of the invention, the receptacle has the form of a dosing pipette, preferably a single dose pipette, which offers a simple and cost-effective manufacturing and a convenient use of the device.

Other embodiments of the inventive device will appear from the dependent claims and the subsequent description.

### BRIEF DESCRIPTION OF THE DRAWINGS

The invention will in the following be more closely described by means of embodiment examples, with reference to the appended drawings. It is shown in:

FIG. 1 a schematic perspective view of a device according to a first embodiment of the present invention,

FIG. 2 a schematic, partly cut lateral view of the device of FIG. 1, and

FIG. 3 a schematic perspective view of a device according to a second embodiment of the present invention.

### DETAILED DESCRIPTION OF PREFERRED EMBODIMENTS

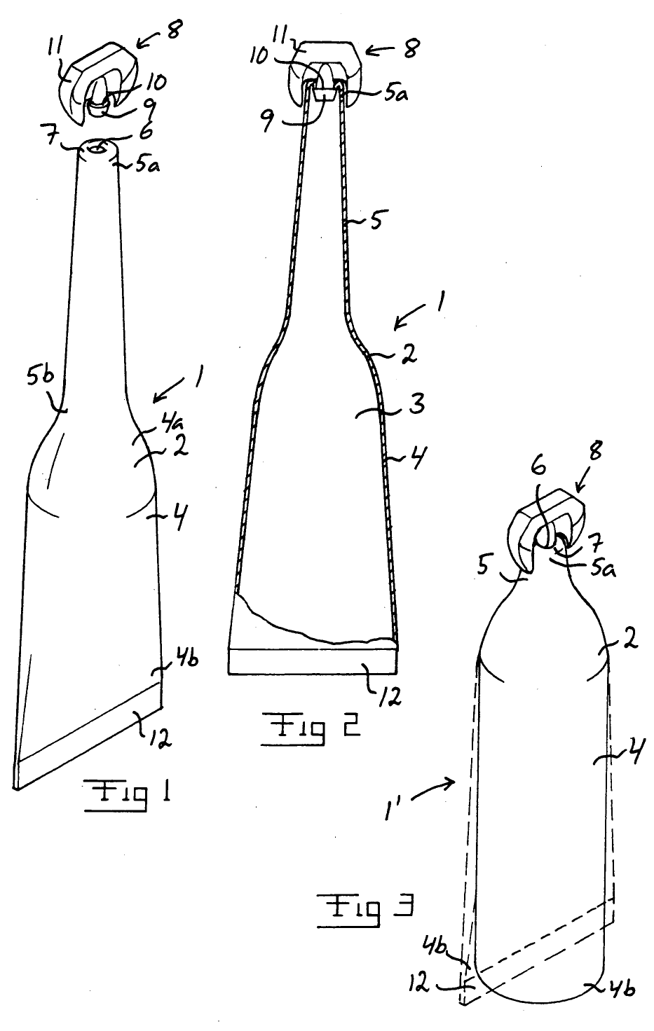

FIGS. 1-3 illustrate two different embodiments of a device 1, 1′ according to the present invention for application of lubricant in a urethra of a patient so as to thereby facilitate the insertion of a medical instrument into the urethra through the urethral orifice. The respective device 1, 1′ comprises a receptacle 2 having a lubricant-containing space 3. The lubricant in said space 3 is preferably lubricant gel but may also have the form of oil, ointment or the like. The receptacle 2 is provided with a gripping part 4 and a sprout 5 connected to the gripping part. The outer end 5a of the sprout is dimensioned to be insertable into the urethra of a patient through the urethral orifice and is provided with an outlet opening 6 connected to the lubricant containing space 3 in the receptacle. The sprout 5 is with advantage conically tapered towards its outer end 5a, as illustrated in FIGS. 1-3. At its outer end 5a, the sprout has a rounded tip 7, which is insertable into the urethra and in which the outlet opening 6 of the sprout is centrally located. At its inner end 5b, the sprout 5 connects to the gripping part 4. The outlet end 6 suitably constitutes the only opening of the lubricant-containing space 3.

The gripping part 4 preferably surrounds at least a part of the lubricant-containing space 3, as illustrated in FIG. 2. In the illustrated embodiments, the lubricant-containing space 3 extends through the gripping part 4 as well as through the sprout 5.

The device 1, 1′ suitably comprises a removable closing member 8, which is arranged to close the outlet opening 6 of the sprout. The closing member 8 is intended to prevent unintentional discharge of lubricant from the receptacle 2. When the device 1, 1′ is to be used, the closing member 8 is removed so that the outlet end 6 of the sprout is uncovered in order to allow discharge of lubricant from the receptacle 2 through the outlet opening. In the illustrated embodiments, the closing member 8 comprises a plug 9, which is insertable into the outlet opening 6 of the sprout and which is secured to the rim of the outlet opening inside the sprout 5 via an external shoulder 10 on the plug in order to obstruct the outlet opening. The illustrated closing member 8 also comprises a gripping member 11 attached to the plug 9, by means of which gripping member the user is able to pull the plug out of the outlet opening 6 of the sprout when the device 1, 1′ is to be used. Other types of closing members than the one here illustrated may of course also be used, such as for instance a tear-off sticker applied over the outlet opening of the sprout.

The receptacle 2 is designed to be grippable and manipulatable by a user by gripping with the fingers about the gripping part 4 of the receptacle, i.e. the receptacle is so designed that the user is able to handle the receptacle in connection with the application of lubricant by gripping and holding the gripping part 4 with the fingers. The gripping part 4 is deformable so that lubricant from the lubricant-containing space 3 can be squeezed out through the outlet opening 6 of the sprout by compression of the gripping part 4 with the fingers, i.e. by the user exerting a pressure with the fingers against the gripping part 4 so that this is compressed. Consequently, the gripping part 4 has such a flexibility that the user is capable of squeezing lubricant out through the outlet opening 6 of the sprout by finger pressing against the gripping part 4.

The gripping part 4 suitably has the form of a compressible, lubricant-containing capsule, preferably with the shape of an elongated tube having a first end 4a connected to the sprout and a closed second end 4b, which can be considered to constitute the rear end of the gripping part. Said rear end 4b of the gripping part is suitably closed by means of a welded seam 12, as illustrated in FIGS. 1-3. In this case, lubricant is introduced into the receptacle 2 through the rear end 4b of the gripping part before this end is closed. The gripping part 4 with open rear end 4b is shown in continuous lines in FIG. 3 and in broken lines after the closure of its rear end 4b.

The receptacle 2 has suitably the form of a dosing pipette, preferably a single dose pipette, as illustrated in FIGS. 1-3. The expression “single dose pipette” here refers to a dosing pipette containing one lubricant dose, for instance about 3-5 grams of lubricant, adapted for one single catheterization.

The receptacle 2 is suitably of plastic material, preferably polyethylene or polypropylene, and is with advantage designed in one piece.

The device 1, 1′ is suitably of disposable type.

The outer end 5a of the sprout, which is insertable into the urethra, has an essentially circular cross-sectional shape and has suitably an external diameter of about 5-8 mm. The outlet end 6 of the sprout is suitably essentially circular with a diameter of about 2-4 mm.

The length of the sprout 5 may be varied as required, but is preferably about 10-60 mm. The female urethral orifice is a little more difficult to reach than the male urethral orifice, and a device 1 (see FIGS. 1 and 2) adapted for women is therefore suitably provided with a longer sprout 5 than a device 1′ (see FIG. 3) adapted for men. The sprout 5 of the receptacle of a device 1 adapted for women suitably has a length of about 40-60 mm, whereas the sprout 5 of a device 1′ adapted for men suitably has a length of about 10-20 mm.

When the device 1, 1′ is to be used in order to apply lubricant in connection with for instance urethral catheterization, the closing member 8 is first removed from the outlet opening 6 of the sprout so that the outlet opening is uncovered. The user then grips the gripping part 4 with his/her fingers and inserts the outer end 5a of the sprout a few millimetres into the urethra of the patient via the urethral orifice so that the tip 7 and outlet opening 6 of the sprout is displaced into the urethra. When the outer end 5a of the sprout with the tip 7 and the outlet opening 6 is in place inside the urethra, the user then compresses the gripping part 4 so that lubricant is squeezed out of the receptacle 2 and into the urethra through the outlet opening 6 of the sprout. After having applied lubricant in the urethra in this manner, the outer end 5a of the sprout is pulled back out of the urethra and the receptacle 2 is removed. A urethral catheter is then in a conventional manner inserted through the urethral orifice into the urethra, which is provided with lubricant, in order to draw urine from the urinary bladder of the patient.

The inventive device may also be used in order to facilitate the insertion into the urethra of another medical instrument than a urethral catheter, such as for instance an endoscope in connection with endoscopy via the urethra. In connection with such an endoscopy, lubricant is first applied in the urethra in the manner described above. An endoscope is then in a conventional manner inserted through the urethral orifice into the urethra that is provided with lubricant.

The invention is of course not in any way limited to the embodiments described above. On the contrary, several possibilities to modifications thereof should be apparent to a person skilled in the art without departing from the basic idea of the invention as defined in the appended claims.

1. A device for application of lubricant, preferably in the form of gel, in an urethra so as to thereby facilitate the insertion of a medical instrument, such an urethral catheter or an endoscope, into the urethra,
wherein
the device (1; 1′) comprises a receptacle (2) having a lubricant-containing space (3), which receptacle is provided with a gripping part (4) and a spout (5) connected to the gripping part;
the outer end (5a) of the spout is dimensioned to be insertable into the urethra of a patient through the urethral orifice and provided with an outlet opening (6) connected to the lubricant-containing space (3) in the receptacle; and the receptacle (2) is grippable and manipulatable by gripping with fingers about the gripping part (4) of the receptacle, the gripping part (4) being deformable so that lubricant from the lubricant-containing space (3) can be squeezed out through the outlet opening (6) of the sprout by compression of the gripping part (4) with the fingers.

2. A device according to claim 1, wherein the receptacle (2) has the form of a dosing pipette, preferably a single dose pipette.

3. A device according to claim 1, wherein the gripping part (4) surrounds at least one part of the lubricant-containing space (3).

4. A device according to claim 1, wherein the gripping part (4) has the form of a compressible, lubricant-containing capsule.

5. A device according to claim 4, wherein the gripping part (4) has the form of an elongated tube having a first end (4a) connected to the sprout (5) and a closed second end (4b).

6. A device according to claim 1, wherein the receptacle (2) is designed in one piece.

7. A device according to claim 1, wherein the outlet opening (6) of the sprout constitutes the only opening of the lubricant-containing space (3).

8. A device according to claim 1, wherein the outlet end (6) of the sprout has a diameter of 2-4 mm.

9. A device according to claim 1, wherein the sprout (5) has a length of 10-50 mm.

10. A device according to claim 1, wherein the outer end (5a) of the sprout that is insertable into the urethra has an external diameter of 5-8 mm.

11. A device according to claim 1, wherein the sprout (5) at its outer end (5a) has a rounded tip (7), which is insertable into the urethra and in which the outlet opening (6) of the sprout is arranged.

12. A device according to claim 2, wherein the gripping part (4) surrounds at least one part of the lubricant-containing space (3).

13. A device according to claim 12, wherein the gripping part (4) has the form of a compressible, lubricant-containing capsule.

14. A device according to claim 2, wherein the gripping part (4) has the form of a compressible, lubricant-containing capsule.

15. A device according to claim 3, wherein the gripping part (4) has the form of a compressible, lubricant-containing capsule.

16. A device according to claim 15, wherein the gripping part (4) has the form of an elongated tube having a first end (4a) connected to the sprout (5) and a closed second end (4b).

17. A device according to claim 14, wherein the gripping part (4) has the form of an elongated tube having a first end (4a) connected to the sprout (5) and a closed second end (4b).

18. A device according to claim 13, wherein the gripping part (4) has the form of an elongated tube having a first end (4a) connected to the sprout (5) and a closed second end (4b).

19. A device according to claim 16, wherein the receptacle (2) is designed in one piece.

20. A device according to claim 17, wherein the receptacle (2) is designed in one piece.
